(19) United States
(12) Patent Application Publication
(10) Pub. No.: US 2023/0313171 A1
(43) Pub. Date: Oct. 5, 2023

(54) METHODS AND COMPOSITIONS FOR REMOVAL OF RNA

(71) Applicant: ADVANCED CELL DIAGNOSTICS, INC., NEWARK, CA (US)

(72) Inventors: Farzaneh Tondnevis, NEWARK, CA (US); Li-Chong Wang, NEWARK, CA (US); Bingqing Zhang, NEWARK, CA (US); Xiao-Jun Ma, NEWARK, CA (US)

(21) Appl. No.: 18/245,660

(22) PCT Filed: Sep. 17, 2021
(86) PCT No.: PCT/US2021/050800

§ 371 Date: Mar. 16, 2023

Related U.S. Application Data
(60) Provisional application No. 63/080,398, filed on Sep. 18, 2020.

(51) Int. Cl. C12N 15/10; C12Q 1/6841

(52) U.S. Cl. CPC C12N 15/1003; C12Q 1/6841

(57) ABSTRACT

The present disclosure provides compositions and methods related to the removal of RNA from a sample, and the preparation of samples for detection of DNA. Such methods are particularly useful to prepare samples for a DNA in situ hybridization assay.

### CROSS-REFERENCE TO RELATED APPLICATIONS

This application claims priority to and the benefit of U.S. Provisional Patent Application No. 63/080,398, filed Sep. 18, 2020, which is incorporated herein by reference in its entirety.

### FIELD

Disclosed herein are methods for removing RNA from a sample, methods for preparing a sample for detection of a target DNA in the sample (e.g., by DNA in situ hybridization), and related compositions and kits.

### BACKGROUND

DNA in situ hybridization (ISH) is a molecular biology technique widely used for detecting a specific sequence in a chromosome, cell or tissue while preserving the chromosomal, cellular and tissue context (Ratan et al., Cureus 9(6):e1325. doi: 10.7759/cureus.1325 (2017)). It has numerous applications in research and diagnostics (Hu et al., Biomark. Res. 2(1):3. doi: 10.1186/2050-7771-2-3 (2014); Ratan et al., supra, 2017; Weier et al., Expert Rev. Mol. Diagn. 2(2):109-119 (2002)). However, current DNA ISH methods can only detect large chromosomal regions (>100 kb) due to their use of large probes and limited sensitivities for shorter sequences. Since the median gene size in the human genome is only 24 kilobases, this means that almost all current DNA ISH probes span more than one gene, which often makes it difficult to draw conclusions at the single gene level. A more sensitive and specific method that allows for visualization of shorter sequences remains a technical challenge.

The recently developed RNA ISH technology called RNAscope™ uses specially designed oligonucleotide probes, sometimes referred to as “double-Z” or ZZ probes, in combination with a branched-DNA-like signal amplification system to reliably detect RNA as small as 1 kilobase at single-molecule sensitivity under standard bright-field microscopy (Anderson et al., J. Cell. Biochem. 117(10):2201-2208 (2016); Wang et al., J. Mol. Diagn. 14(1):22-29 (2012)). Such a probe design improves the specificity of signal amplification because signal amplification can occur only when both probes in each pair bind to their intended target. However, a direct application of RNAscope™ for DNA detection is hampered by unwanted RNA detection because RNAscope™ probes cannot discriminate DNA from RNA targets. Although RNA can be eliminated by enzymatic methods (e.g., RNase A) and chemical methods (e.g., NaOH), adding these steps can cause significant degradation of nuclear and cellular morphology and can compromise DNA detection.

### SUMMARY

The present disclosure provides a method of removing RNA from a sample, the method comprising: contacting the sample with an effective amount of sodium metasilicate.

In some embodiments, the sample is a biological sample. In some embodiments, the sample comprises cultured cells. In some embodiments, the sample is a tissue specimen or is derived from a tissue specimen. In some embodiments, sample is a formalin fixed paraffin embedded tissue specimen. In some embodiments, the method further comprises performing a deparaffinization step prior to contacting the sample with the sodium metasilicate. In some embodiments, the sample is a blood sample or is derived from a blood sample. In some embodiments, the sample is a cytological sample or is derived from a cytological sample.

In some embodiments, the sodium metasilicate is in an aqueous solution. In some embodiments, the concentration of the sodium metasilicate in the solution is about 50 mM to about 200 mM. In some embodiments, the concentration of the sodium metasilicate in the solution is about 100 mM. In some embodiments, the solution of sodium metasilicate has a pH of about 12 to about 14. In some embodiments, the solution of sodium metasilicate has a pH of about 12.5 to about 13.0.

In some embodiments, the method further comprises heating the sample after the contacting step. In some embodiments, the sample is heated to a temperature of about 35° C. to about 45° C. In some embodiments, the sample is heated to a temperature of about 40° C. In some embodiments, the method comprises heating the sample for about 30 minutes to about 60 minutes. In some embodiments, the method comprises heating the sample for about 45 minutes.

In some embodiments, the method further comprises washing the sample after the contacting step.

In some embodiments, the method further comprises detecting a target DNA in the sample after the contacting step. In some embodiments, the target DNA is detected by DNA in situ hybridization.

In some embodiments, the sample morphology is substantially unchanged following the contacting step. In some embodiments, RNA levels are reduced by at least 90% in the sample. In some embodiments, the RNA removed from the sample comprises mRNA.

The present disclosure also provides a method of preparing a biological sample for detection of a target DNA in the sample, comprising: contacting the sample with sodium metasilicate.

In some embodiments, the biological sample comprises cultured cells. In some embodiments, the biological sample is a tissue specimen or is derived from a tissue specimen. In some embodiments, the biological sample is a formalin fixed paraffin embedded tissue specimen. In some embodiments, the method further comprises performing a deparaffinization step prior to contacting the biological sample with the sodium metasilicate. In some embodiments, the biological sample is a blood sample or is derived from a blood sample. In some embodiments, the biological sample is a cytological sample or is derived from a cytological sample.

In some embodiments, the sodium metasilicate is in an aqueous solution. In some embodiments, the concentration of the sodium metasilicate in the solution is about 50 mM to about 200 mM. In some embodiments, the concentration of the sodium metasilicate in the solution is about 100 mM. In some embodiments, the solution of sodium metasilicate has a pH of about 12 to about 14. In some embodiments, the solution of sodium metasilicate has a pH of about 12.5 to about 13.0.

In some embodiments, the method further comprises heating the sample after the contacting step. In some embodiments, the sample is heated to a temperature of about 35° C. to about 45° C. In some embodiments, the sample is heated to a temperature of about 40° C. In some embodiments, the method comprises heating the sample for about 30 minutes to about 60 minutes. In some embodiments, the method comprises heating the sample for about 45 minutes.

In some embodiments, the method further comprises washing the sample after the contacting step.

In some embodiments, the method further comprises detecting a target DNA in the sample after the contacting step. In some embodiments, the target DNA is detected by DNA in situ hybridization.

In some embodiments, the sample morphology is substantially unchanged following contacting of the sample with the sodium metasilicate. In some embodiments, RNA levels are reduced by at least 90% in the sample. In some embodiments, the RNA removed from the sample comprises mRNA.

The present disclosure also provides a composition comprising: sodium metasilicate; and a sample comprising a plurality of cells.

In some embodiments, the sample is a tissue specimen or is derived from a tissue specimen. In some embodiments, the sample is a formalin fixed paraffin embedded tissue specimen or is derived from a formalin fixed paraffin embedded tissue specimen. In some embodiments, the sample is a blood sample or is derived from a blood sample. In some embodiments, the sample is a cytological sample or is derived from a cytological sample.

The present disclosure also provides a kit comprising: sodium metasilicate; and one or more probes or reagents for detecting a target DNA in a sample.

In some embodiments, the kit comprises one or more target probes capable of hybridizing to the target DNA in the sample. In some embodiments, the kit comprises one or more reagents for detecting DNA in the sample, wherein the reagents are selected from a hybridization buffer, dextran sulfate, formamide, dithiothreitol (DTT), sodium chloride and sodium citrate (SSC), EDTA, Denhardt's solution, a fluorescent label, a chromogenic label, dNTPs, single-stranded DNA, tRNA, polyA, an initiator oligonucleotide, or any combination thereof.

In some embodiments, the kit further comprises a signal generating complex capable of hybridizing to the one or more target probes. In some embodiments, the signal generating complex comprises a label probe, and optionally, one or more of an amplifier, a pre-amplifier, and a pre-pre-amplifier.

In some embodiments, the kit further comprises a calibrator or control polynucleotide.

In some embodiments, the sodium metasilicate is in an aqueous solution. In some embodiments, the concentration of the sodium metasilicate in the solution is about 50 mM to about 200 mM. In some embodiments, the concentration of the sodium metasilicate in the solution is about 100 mM. In some embodiments, the solution of sodium metasilicate has a pH of about 12 to about 14. In some embodiments, the solution of sodium metasilicate has a pH of about 12.5 to about 13.0.

In some embodiments, the kit further comprises instructions for carrying out a DNA in situ hybridization assay.

The present disclosure also provides a kit comprising: sodium metasilicate; and instructions for removing RNA from a sample using the sodium metasilicate.

In some embodiments, the sodium metasilicate is in an aqueous solution. In some embodiments, the concentration of the sodium metasilicate in the solution is about 50 mM to about 200 mM. In some embodiments, the concentration of the sodium metasilicate in the solution is about 100 mM. In some embodiments, the solution of sodium metasilicate has a pH of about 11 to about 14. In some embodiments, the solution of sodium metasilicate has a pH of about 12.5 to about 13.0.

Other aspects and embodiments of the disclosure will be apparent in light of the following detailed description.

### BRIEF DESCRIPTION OF THE DRAWINGS

FIG. 1 shows images of cells from which RNA was removed using the traditional enzymatic approach with RNase A.

FIG. 2 shows images of HeLa cells from which RNA was removed using 100 mM sodium metasilicate (pH 12.8); the solution was added to FFPE cells after deparaffinization. Various RNA targets, including those with low to very high expression levels, were detected using an RNAscope™ assay.

FIG. 3 shows images of various cell and tissue samples from which RNA was removed using sodium metasilicate (pH 12.8); the solution was added to FFPE cells after deparaffinization. The targets were detected using an RNAscope™-based DNA ISH assay.

FIG. 4 shows fluorescence images of HeLa cells from which RNA was removed using sodium metasilicate (pH 12.8); the staining pattern using the RNAscope™ assay before and after RNA removal treatment for the FFPE sample stained with MALAT1 antisense probe is shown.

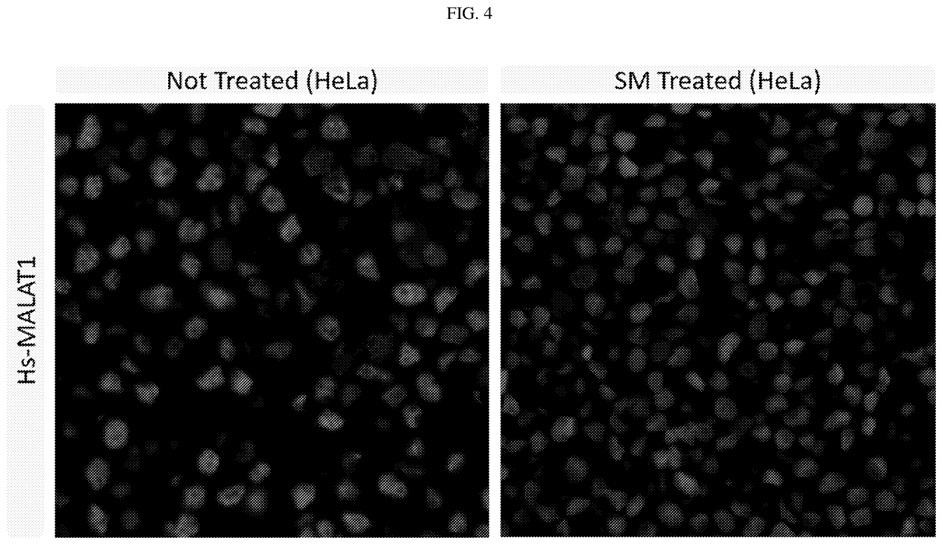

### DETAILED DESCRIPTION

Disclosed herein are methods for rapid and efficient removal of RNA molecules from various samples, which enables specific detection of a DNA target of interest without cross-detection of RNA. This method of RNA removal provides high levels of efficiency with RNA removal rates in some embodiments of ≥90% (e.g., ≥95%), even when tested on RNA targets with very high expression levels. Additionally, there is little to no damage to the nuclear and cellular morphology as compared to methods using RNase enzymes or other chemicals such as sodium hydroxide.

The disclosed methods include application of a solution of sodium metasilicate during the pretreatment portion of a DNA ISH assay. Preservation of morphology is of great importance in the context of an in situ hybridization assay, and sodium metasilicate does not have adverse effects on the cellular and nuclear morphology while effectively removing RNA molecules from various samples.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the present disclosure may be readily combined, without departing from the scope or spirit of the embodiments provided herein. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The terms “nucleic acid” and “polynucleotide” are used interchangeably herein to describe a polymer of any length composed of nucleotides, e.g., deoxyribonucleotides or ribonucleotides, or compounds produced synthetically, which can hybridize with naturally occurring nucleic acids in a sequence specific manner analogous to that of two naturally occurring nucleic acids, e.g., can participate in Watson-Crick base pairing interactions. As used herein in the context of a polynucleotide sequence, the term “bases” (or “base”) is synonymous with “nucleotides” (or “nucleotide”), i.e., the monomer subunit of a polynucleotide. The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like. “Analogues” refer to molecules having structural features that are recognized in the literature as being mimetics, derivatives, having analogous structures, or other like terms, and include, for example, polynucleotides incorporating non-natural nucleotides, nucleotide mimetics such as 2′-modified nucleosides, peptide nucleic acids, oligomeric nucleoside phosphonates, and any polynucleotide that has added substituent groups, such as protecting groups or linking moieties.

The term “probe” as used herein refers to a capture agent that is directed to a specific target nucleic acid sequence. Accordingly, each probe of a probe set has a respective target nucleic acid sequence. In some embodiments, the probe provided herein is a “nucleic acid probe” or “oligonucleotide probe” which refers to a nucleic acid capable of binding to a target nucleic acid of complementary sequence, usually through complementary base pairing by forming hydrogen bonds. As used herein, a probe may include natural (e.g., A, G, C, or T) or modified bases (7-deazaguanosine, inosine, etc.). In addition, the bases in a probe may be joined by a linkage other than a phosphodiester bond, so long as it does not interfere with hybridization. The probes can be directly or indirectly labeled with tags, for example, chromophores, lumiphores, or chromogens. By assaying for the presence or absence of the probe, one can detect the presence or absence of a target nucleic acid of interest.

The term “gene” refers to a nucleic acid (e.g., DNA) sequence that comprises coding sequences for the production of a polypeptide, precursor, or RNA (e.g., rRNA, tRNA, sRNA, microRNA, lincRNA). The polypeptide can be encoded by a full-length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the terms “complementary” or “complementarity” are used in reference to polynucleotides (e.g., a sequence of nucleotides such as an oligonucleotide or a target nucleic acid) related by the base-pairing rules. For example, for the sequence “5′-A-G-T-3′” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. Either term may also be used in reference to individual nucleotides, especially within the context of polynucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand.

In some contexts, the term “complementarity” and related terms (e.g., “complementary”, “complement”) refers to the nucleotides of a nucleic acid sequence that can bind to another nucleic acid sequence through hydrogen bonds, e.g., nucleotides that are capable of base pairing, e.g., by Watson-Crick base pairing or other base pairing. Nucleotides that can form base pairs, e.g., that are complementary to one another, are the pairs: cytosine and guanine, thymine and adenine, adenine and uracil, and guanine and uracil. The percentage complementarity need not be calculated over the entire length of a nucleic acid sequence. The percentage of complementarity may be limited to a specific region of which the nucleic acid sequences that are base-paired, e.g., starting from a first base-paired nucleotide and ending at a last base-paired nucleotide. The complement of a nucleic acid sequence as used herein refers to an oligonucleotide which, when aligned with the nucleic acid sequence such that the 5′ end of one sequence is paired with the 3′ end of the other, is in “antiparallel association.” Certain bases not commonly found in natural nucleic acids may be included in the nucleic acids of the present disclosure and include, for example, inosine and 7-deazaguanine. Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

Thus, in some embodiments, “complementary” refers to a first nucleobase sequence that is at least 60%, 65%, 70%, 75%, 80%, 85%, 90%, 95%, 97%, 98%, or 99% identical to the complement of a second nucleobase sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases, or that the two sequences hybridize under stringent hybridization conditions. “Fully complementary” means each nucleobase of a first nucleic acid is capable of pairing with each nucleobase at a corresponding position in a second nucleic acid. For example, in certain embodiments, an oligonucleotide wherein each nucleobase has complementarity to a nucleic acid has a nucleobase sequence that is identical to the complement of the nucleic acid over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases.

The term “sample” as used herein relates to a material or mixture of materials containing one or more components of interest. The term “sample” includes “biological sample” which refers to a sample obtained from a biological subject, including a sample of biological tissue or fluid origin, obtained, reached, or collected in vivo or in situ. Such samples can be, but are not limited to, organs, tissues, and cells isolated from a mammal. Exemplary biological samples include but are not limited to cell lysate, a cell culture, a cell line, a tissue, oral tissue, gastrointestinal tissue, an organ, an organelle, a biological fluid, a blood sample, a urine sample, a skin sample, and the like.

The terms “detecting” as used herein generally refer to any form of measurement, and include determining whether an element is present or not. This term includes quantitative and/or qualitative determinations.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. Methods of RNA Removal and Preparing Samples for Detection of DNA

Disclosed herein are methods of removing RNA from a sample, comprising contacting the sample with an effective amount of sodium metasilicate. Also disclosed herein are methods of preparing a sample for detection of a target DNA, comprising contacting the sample with an effective amount of sodium metasilicate.

Sodium metasilicate (Na₂SiO₃) is an ionic compound consisting of sodium cations and metasilicate anions. It is commercially available from a variety of suppliers, such as Alfa Chemistry, Acros Organics, Fisher Scientific, Sigma-Aldrich, VWR, and others. It is available as the anhydrous form (in which the metasilicate anion is in polymeric form, —(SiO₃²⁻—)ₙ), and as a hydrated form (e.g., sodium metasilicate pentahydrate and sodium metasilicate nonahydrate). In some embodiments, of the methods, compositions, and kits described herein, the sodium metasilicate is anhydrous sodium metasilicate. When a sample containing RNA is contacted with sodium metasilicate, the sodium metasilicate treatment creates an alkaline condition in which there is breakdown of RNA chains through a series of chain reactions, which leads to eventual hydrolysis of the RNA molecules.

In some embodiments, the sample is contacted with an aqueous solution of sodium metasilicate. The aqueous solution may have a concentration of sodium metasilicate of about 50 mM to about 200 mM, for example, about 50 mM, about 55 mM, about 60 mM, about 65 mM, about 70 mM, about 75 mM, about 80 mM, about 85 mM, about 90 mM, about, 95 mM, about 100 mM, about, 105 mM, about 110 mM, about 115 mM, about 120 mM, about 125 mM, about 130 mM, about 135 mM, about 140 mM, about 145 mM, about 150 mM, about 155 mM, about 160 mM, about 165 mM, about 170 mM, about 175 mM, about 180 mM, about 185 mM, about 190 mM, about 195 mM, or about 200 mM. In some embodiments, the aqueous solution has a concentration of sodium metasilicate of about 100 mM.

In some embodiments, the aqueous solution of sodium metasilicate has a pH of about 12 to about 14, or about 12.5 to about 13.5, or about 12.5 to about 13.0. For example, in some embodiments, the aqueous solution of sodium metasilicate has a pH of about 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, or 14.0. In some embodiments, the aqueous solution of sodium metasilicate has a pH of about 12.8.

The sodium metasilicate treatment can be used to remove RNA from a variety of samples, including biological samples. In some embodiments, the sample comprises cultured cells. In some embodiments, the sample is a tissue specimen or is derived from a tissue specimen. In some embodiments, the sample is a blood sample or is derived from a blood sample. In some embodiments, the sample is a cytological sample or is derived from a cytological sample. In another embodiment, the biological sample is an exosome.

Tissue specimens include, for example, tissue biopsy samples. Blood samples include, for example, blood samples taken for diagnostic purposes. In the case of a blood sample, the blood can be directly analyzed, such as in a blood smear, or the blood can be processed, for example, lysis of red blood cells, isolation of PBMCs or leukocytes, isolation of target cells, and the like. Similarly, a tissue specimen can be processed, for example, the tissue specimen minced and treated physically or enzymatically to disrupt the tissue into individual cells or cell clusters. Additionally, a cytological sample can be processed to isolate cells or disrupt cell clusters, if desired. Thus, the tissue, blood and cytological samples can be obtained and processed using methods well known in the art. The methods of the disclosure can be used in diagnostic applications to identify the presence or absence of pathological cells based on the presence or absence of a nucleic acid target that is a biomarker indicative of a pathology.

The sample for use in the methods provided herein is generally a biological sample or tissue sample. Such a sample can be obtained from a subject, including a sample of biological tissue or fluid origin that is collected from an individual or some other source of biological material such as biopsy, autopsy, or forensic materials. A biological sample also includes samples from a region of a subject containing or suspected of containing precancerous or cancer cells or tissues, for example, a tissue biopsy, including fine needle aspirates, blood sample or cytological specimen. Such samples can be, but are not limited to, organs, tissues, tissue fractions, cells, and/or exosomes isolated from an organism such as a mammal. Exemplary biological samples include, but are not limited to, a cell culture, including a primary cell culture, a cell line, a tissue, an organ, an organelle, a biological fluid, and the like. Additional biological samples include but are not limited to a skin sample, tissue biopsies, including fine needle aspirates, cytological samples, stool, bodily fluids, including blood and/or serum samples, saliva, semen, and the like. Such samples can be used for medical or veterinary diagnostic purposes.

Collection of cytological samples for analysis by methods provided herein are well known in the art (see, for example, Dey, “Cytology Sample Procurement, Fixation and Processing” in Basic and Advanced Laboratory Techniques in Histopathology and Cytology pp. 121-132, Springer, Singapore (2018); “Non-Gynecological Cytology Practice Guideline” American Society of Cytopathology, Adopted by the ASC executive board Mar. 2, 2004).

For example, methods for processing samples for analysis of cervical tissue, including tissue biopsy and cytology samples, are well known in the art (see, for example, Cecil Textbook of Medicine, Bennett and Plum, eds., 20th ed., WB Saunders, Philadelphia (1996); Colposcopy and Treatment of Cervical Intraepithelial Neoplasia: A Beginner's Manual, Sellors and Sankaranarayanan, eds., International Agency for Research on Cancer, Lyon, France (2003); Kalaf and Cooper, J. Clin. Pathol. 60:449-455 (2007); Brown and Trimble, Best Pract. Res. Clin. Obstet. Gynaecol. 26:233-242 (2012); Waxman et al., Obstet. Gynecol. 120:1465-1471 (2012); Cervical Cytology Practice Guidelines TOC, Approved by the American Society of Cytopathology (ASC) Executive Board, Nov. 10, 2000)).

In some embodiments, the sample is a tissue specimen. In some embodiments, the sample is a formalin-fixed paraffin-embedded (FFPE) tissue specimen. In some embodiments, the tissue specimen is fresh frozen. In some embodiments, the tissue specimen is prepared with a fixative other than formalin. In some embodiments, the fixative other than formalin is selected from the group consisting of ethanol, methanol, formal calcium, formal saline, zinc formalin, Zenker's fixative, Helly's fixative, B-5 fixative, Bouin's solution, Hollande's fixative, Gendre's solution, Clarke's solution, Carnoy's solution, Methacarn, Alcoholic formalin, formol acetic alcohol, and I.B.F. tissue fixative.

When the sample is a FFPE tissue specimen, the method may further comprise a deparaffinization step (also known as dewaxing) prior to contacting the sample with the sodium metasilicate. Deparaffinization is typically performed washing the specimen with a non-polar solvent, such as xylene, a mineral oil, or other suitable hydrocarbon-based solvent. The washing step with the non-polar solvent is typically performed multiple times. An optional heating step to melt the wax can be performed prior to washing. After the washing steps with the non-polar solvent, the non-polar solvent can be removed by successive washing steps with graded concentrations of ethanol, e.g., first with a 50:50 mixture of xylene and ethanol, followed by washing with solutions having successively lower concentrations of ethanol (e.g., 100% ethanol, then one or more washes with solutions of 95% ethanol, 90% ethanol, 85% ethanol, 80% ethanol, 75% ethanol, 70% ethanol, 65% ethanol, 60% ethanol, 55% ethanol, and/or 50% ethanol, or any combination thereof), followed by one or more final washes with water.

The step of contacting the sample with the sodium metasilicate can be conducted by any suitable means. For example, if the sample is a liquid sample (e.g., a sample of cultured cells in solution, a blood sample, or a liquid cytological sample), an aqueous solution of sodium metasilicate can be added to the sample followed by appropriate mixing. If the sample comprises fixed cells (e.g., on a slide), an aqueous solution of sodium metasilicate can be applied to the fixed cells and the sample can be incubated for a certain period of time.

In the disclosed methods, the sample can be heated after it is contacted with the sodium metasilicate. In some embodiments, the sample is heated to a temperature of about 35° C. to about 45° C., for example about 35° C., about 36° C., about 37° C., about 38° C., about 39° C., about 40° C., about 41° C., about 42° C., about 43° C., about 44° C., or about 45° C. In some embodiments, the sample is heated to a temperature of about 40° C. In some embodiments, the sample is heated for a time of about 30 minutes to about 60 minutes, e.g., about 30, 35, 40, 45, 50, 55, or 60 minutes. In some embodiments, the sample is heated for about 45 minutes.

In some embodiments, the method further comprises washing the sample after the contacting step. The washing step removes excess sodium metasilicate from the sample, which may be desirable in embodiments in which a component of the sample is detected after the RNA is removed from the sample. In some embodiments, the sample is washed with water, or an aqueous solution comprising one or more components such as buffers, salts, or the like.

The methods can remove different types of RNA that exist in a sample (e.g., a cell), including messenger RNA (mRNA), transfer RNA (tRNA), ribosomal RNA (rRNA), small nuclear RNA (snRNA), small non-coding RNA (sncRNA), microRNA (miRNA), PIWI-interacting RNA (piRNA), small interfering RNA (siRNA), antisense RNA (aRNA), long non-coding RNA (lncRNA), and others. In some embodiments, the methods remove mRNA.

Advantageously, the methods of removing RNA from samples described herein do not substantially affect the cellular or nuclear morphology. This is particularly important in the context of an in situ hybridization assay. Accordingly, in some embodiments, the sample morphology (e.g., cellular morphology or nuclear morphology) is substantially unchanged following the step of contacting the sample with the sodium metasilicate. As those skilled in the art appreciate, morphology is typically assessed by inspection of nuclei and cytoplasm intactness post-hematoxylin staining, and by inspection of a normal vs. shrunken appearance of cells and nuclei. General sample detachment can also be investigated.

The methods disclosed herein can reduce RNA levels in the sample by at least 90%, e.g., compared to a sample that has not been contacted with the sodium metasilicate. For example, in some embodiments, RNA levels in the sample are reduced by at least 90%, at least 91%, at least 92%, at least 93%, at least 94%, at least 95%, at least 96%, at least 97%, at least 98%, or at least 99%.

Embodiments of the present disclosure also provide a use of a composition comprising sodium metasilicate for removal of RNA from a sample. Other embodiments provide a use a composition comprising sodium metasilicate for the preparation of a biological sample for detection of a target DNA in the sample.

### 3. Methods of Detecting DNA

The disclosed methods of removing RNA from a sample can be used with any sample for which it would be desirable to remove RNA. The methods are particularly suitable to prepare a biological sample for detection of a target DNA in the sample, especially those in which the DNA detection method can cross-detect RNA. In some embodiments, the methods can be used to prepare a sample for detection of DNA in an in situ hybridization (ISH) assay.

Methods for in situ detection of nucleic acids are well known to those skilled in the art (see, for example, US 2008/0038725; US 2009/0081688; Hicks et al., J. Mol. Histol. 35:595-601 (2004)). As used herein, “in situ hybridization” or “ISH” refers to a type of hybridization that uses a directly or indirectly labeled complementary DNA or RNA strand, such as a probe, to bind to and localize a specific nucleic acid in a sample, in particular a portion or section of tissue or cells (in situ). The probe types can be double stranded DNA (dsDNA), single stranded DNA (ssDNA), single stranded complimentary RNA (sscRNA), messenger RNA (mRNA), micro RNA (miRNA), ribosomal RNA, mitochondrial RNA, and/or synthetic oligonucleotides.

In some embodiments, the in situ hybridization provided herein comprises providing at least one set of one or more target probe(s) capable of hybridizing to said target nucleic acid; providing a signal-generating complex capable of hybridizing to said set of one or more target probe(s), said signal-generating complex comprises a nucleic acid component capable of hybridizing to said set of one or more target probe(s) and a label probe; hybridizing said target nucleic acid to said set of one or more target probe(s); and capturing the signal-generating complex to said set of one or more target probe(s) and thereby capturing the signal-generating complex to said target nucleic acid.

In some embodiments, each set of one or more target probe(s) comprises a single probe. In other embodiments, each set of one or more target probe(s) comprises two probes. In yet other embodiments, each set of one or more target probe(s) comprises more than two probes.

In some embodiments, when each set of target probes comprises a single target probe, a signal-generating complex is formed when the single target probe is bound to the target nucleic acid. In other embodiments, when each set of target probes comprise two target probes, a signal-generating complex is formed when both members of a target probe pair are bound to the target nucleic acid.

In some specific embodiments, the DNA ISH used herein is based on RNAscope®. It uses the RNAscope core technology, with modifications added in the “pretreatment” steps to optimize for DNA detection. Specifically, in some embodiments, a DNA denaturation step using formamide at an elevated temperature (e.g., 70% formamide at 80° C.) is added before probe hybridization, which is described in more detail in, e.g., U.S. Pat. Nos. 7,709,198, 8,604,182, and 8,951,726, which are incorporated herein by reference in their entireties. Specifically, RNAscope® involves use of specially designed oligonucleotide probes in combination with a branched-DNA-like signal-generating complex to reliably detect RNA as small as 1 kilobase at single-molecule sensitivity under standard bright-field microscopy (Anderson et al., J. Cell. Biochem. 117(10):2201-2208 (2016); Wang et al., J. Mol. Diagn. 14(1):22-29 (2012); each of which is incorporated herein by reference in its entirety). Such a probe design greatly improves the specificity of signal amplification because only when both probes in each pair bind to their intended target can signal amplification occur. Use of RNAscope® to detect DNA in a sample is possible in the methods disclosed herein because the RNA is removed from the sample using sodium metasilicate, preventing significant cross-detection of RNA.

In one embodiment, the DNA ISH used herein is based on BaseScope™, which is described in more detail in, e.g., US Patent Publication No. 2013/0171621, and PCT Publication No. WO 2011/094669, which are incorporated herein by reference in their entireties. Specifically, BaseScope™ includes the use of specially designed oligonucleotide probes, sometimes referred to as “double-Z” or ZZ probes, in combination with a branched-DNA-like signal amplification system to reliably detect target nucleic acids with single-molecule sensitivity under standard bright-field microscopy. Such a probe design greatly improves the specificity of signal amplification because only when both probes in each pair bind to their intended target can signal amplification occur, enabling the detecting of biological events in cells and in situ using a single Z probe pair. As with RNAscope®, use of BaseScope™ to detect DNA in a sample is possible in the methods disclosed herein because the RNA is removed from the sample using sodium metasilicate, preventing any cross-detection of RNA.

In another embodiment, the ISH methods of the present disclosure include the use of probes that form stable DNA hairpins, along with a DNA initiator probe. These probes can be used to detect a target nucleic acid using a hybridization chain reaction (HCR) mechanism. The addition of an initiator strand of DNA to the stable mixture of two hairpin species triggers a chain reaction of hybridization events between the hairpins, which is used to amplify a detectable signal (see, e.g., Dirks, R. M. and Pierce, N. A. Triggered amplification by hybridization chain reaction. Proc. Natl. Acad. Sci. USA 101, 15275-15278 (2004)).

In another embodiment, the DNA ISH used herein is described in PCT Appln. No. PCT/US2020/022010. This assay uses a probe design strategy that provides specific detection of double stranded DNA using the principles of RNAscope®, where each probe pair binds to both strands of the double stranded DNA. Like RNAscope® probes, each probe contains a sequence segment that binds to a specific sequence in the target. For double stranded nucleic acid detection, for example, DNA detection, two probes bind to adjacent sites on opposite strands in the target double stranded nucleic acid. Only when both probes bind to their respective target sites simultaneously can a full binding site for the signal amplification molecule (for example, a pre-amplifier or a pre-pre-amplifier) be formed, leading to successful signal amplification and detection. The target DNA has any suitable length, from about 1 kilobase (kb) to hundreds of kb or even larger (e.g., a full chromosome).

In some embodiments, each target probe comprises a target (T) section and a label (L) section, wherein the T section is a nucleic acid sequence complementary to a section on the target nucleic acid and the L section is a nucleic acid sequence complementary to a section on the nucleic acid component of the signal-generating complex, and wherein the T sections of the one or more target probe(s) are complementary to non-overlapping regions of the target nucleic acid, and the L sections of the one or more target probe(s) are complementary to non-overlapping regions of the nucleic acid component of the generating complex.

In some embodiments, one set of one or more target probe(s) is used to detect a target nucleic acid. In other embodiments, two or more sets of one or more target probe(s) are used to detect a target nucleic acid. In some embodiments, two, three, four, five, six, seven, eight, nine, ten, more than ten, more than 15, or more than 20 sets of one or more target probe(s) are used to detect a target nucleic acid. In some embodiments, the method provided herein is for detecting multiple nucleic acid targets.

As used herein, a “target probe” is a polynucleotide that is capable of hybridizing to a target nucleic acid and capturing or binding a label probe or signal-generating complex (SGC) component to that target nucleic acid. The target probe can hybridize directly to the label probe, or it can hybridize to one or more nucleic acids that in turn hybridize to the label probe; for example, the target probe can hybridize to an amplifier, a pre-amplifier or a pre-pre-amplifier in an SGC. The target probe thus includes a first polynucleotide sequence that is complementary to a polynucleotide sequence of the target nucleic acid and a second polynucleotide sequence that is complementary to a polynucleotide sequence of the label probe, amplifier, pre-amplifier, pre-pre-amplifier, or the like. The target probe is generally single stranded so that the complementary sequence is available to hybridize with a corresponding target nucleic acid, label probe, amplifier, pre-amplifier or pre-pre-amplifier. In some embodiments, the target probes are provided as a pair.

As used herein, the term “label probe” refers to an entity that binds to a target molecule, directly or indirectly, generally indirectly, and allows the target to be detected. A label probe (or “LP”) contains a nucleic acid binding portion that is typically a single stranded polynucleotide or oligonucleotide that comprises one or more labels which directly or indirectly provides a detectable signal. The label can be covalently attached to the polynucleotide, or the polynucleotide can be configured to bind to the label. For example, a biotinylated polynucleotide can bind a streptavidin-associated label. The label probe can, for example, hybridize directly to a target nucleic acid. In general, the label probe can hybridize to a nucleic acid that is in turn hybridized to the target nucleic acid or to one or more other nucleic acids that are hybridized to the target nucleic acid. Thus, the label probe can comprise a polynucleotide sequence that is complementary to a polynucleotide sequence, particularly a portion, of the target nucleic acid. Alternatively, the label probe can comprise at least one polynucleotide sequence that is complementary to a polynucleotide sequence in an amplifier, pre-amplifier, or pre-pre-amplifier in a SGC. In some embodiments, the SGC provided herein comprises additional components such an amplifier, a pre-amplifier, and/or a pre-pre-amplifier.

As used herein, an “amplifier” is a molecule, typically a polynucleotide, that is capable of hybridizing to multiple label probes. Typically, the amplifier hybridizes to multiple identical label probes. The amplifier can also hybridize to a target nucleic acid, to at least one target probe of a pair of target probes, to both target probes of a pair of target probes, or to nucleic acid bound to a target probe such as an amplifier, pre-amplifier or pre-pre-amplifier. For example, the amplifier can hybridize to at least one target probe and to a plurality of label probes, or to a pre-amplifier and a plurality of label probes. The amplifier can be, for example, a linear, forked, comb-like, or branched nucleic acid. As described herein for all polynucleotides, the amplifier can include modified nucleotides and/or nonstandard internucleotide linkages as well as standard deoxyribonucleotides, ribonucleotides, and/or phosphodiester bonds. Suitable amplifiers are described, for example, in U.S. Pat. Nos. 5,635,352, 5,124,246, 5,710,264, 5,849,481, and 7,709,198 and U.S. publications 2008/0038725 and 2009/0081688, each of which is incorporated by reference in its entirety.

As used herein, a “pre-amplifier” is a molecule, typically a polynucleotide, that serves as an intermediate binding component between one or more target probes and one or more amplifiers. Typically, the pre-amplifier hybridizes simultaneously to one or more target probes and to a plurality of amplifiers. Exemplary pre-amplifiers are described, for example, in U.S. Pat. Nos. 5,635,352, 5,681,697 and 7,709,198 and U.S. publications 2008/0038725, 2009/0081688 and 2017/0101672, each of which is incorporated by reference in its entirety.

As used herein, a “pre-pre-amplifier” is a molecule, typically a polynucleotide, that serves as an intermediate binding component between one or more target probes and one or more pre-amplifiers. Typically, the pre-pre-amplifier hybridizes simultaneously to one or more target probes and to a plurality of pre-amplifiers. Exemplary pre-pre-amplifiers are described, for example, in 2017/0101672, which is incorporated by reference in its entirety.

A label is typically used in an in situ hybridization assay for detecting target nucleic acid. As used herein, a “label” is a moiety that facilitates detection of a molecule. Common labels include fluorescent, luminescent, light-scattering, and/or colorimetric labels. Suitable labels include enzymes, and fluorescent and chromogenic moieties, as well as radionuclides, substrates, cofactors, inhibitors, chemiluminescent moieties, magnetic particles, rare earth metals, metal isotopes, and the like. In a particular embodiment, the label is an enzyme. Exemplary enzyme labels include, but are not limited to horseradish peroxidase (HRP), alkaline phosphatase (AP), β-galactosidase, glucose oxidase, and the like, as well as various proteases. Other labels include, but are not limited to, fluorophores, dinitrophenyl (DNP), and the like. Labels are well known to those skilled in the art, as described, for example, in Hermanson, Bioconjugate Techniques, Academic Press, San Diego (1996), and U.S. Pat. Nos. 3,817,837; 3,850,752; 3,939,350; 3,996,345; 4,277,437; 4,275,149; and 4,366,241. Many labels are commercially available and can be used in methods and assays of the disclosure, including detectable enzyme/substrate combinations (Pierce, Rockford IL; Santa Cruz Biotechnology, Dallas TX; Life Technologies, Carlsbad CA). In a particular embodiment of the disclosure, the enzyme can utilize a chromogenic or fluorogenic substrate to produce a detectable signal, as described herein. Exemplary labels are described herein.

Any of a number of enzymes or non-enzyme labels can be utilized so long as the enzymatic activity or non-enzyme label, respectively, can be detected. The enzyme thereby produces a detectable signal, which can be utilized to detect a target nucleic acid. Particularly useful detectable signals are chromogenic or fluorogenic signals. Accordingly, particularly useful enzymes for use as a label include those for which a chromogenic or fluorogenic substrate is available. Such chromogenic or fluorogenic substrates can be converted by enzymatic reaction to a readily detectable chromogenic or fluorescent product, which can be readily detected and/or quantified using microscopy or spectroscopy. Such enzymes are well known to those skilled in the art, including but not limited to, horseradish peroxidase, alkaline phosphatase, β-galactosidase, glucose oxidase, and the like (see Hermanson, Bioconjugate Techniques, Academic Press, San Diego (1996)). Other enzymes that have well known chromogenic or fluorogenic substrates include various peptidases, where chromogenic or fluorogenic peptide substrates can be utilized to detect proteolytic cleavage reactions. The use of chromogenic and fluorogenic substrates is also well known in bacterial diagnostics, including but not limited to the use of α- and β-galactosidase, β-glucuronidase, 6-phospho-β-D-galactoside 6-phosphogalactohydrolase, β-glucosidase, α-glucosidase, amylase, neuraminidase, esterases, lipases, and the like (Manafi et al., Microbiol. Rev. 55:335-348 (1991)), and such enzymes with known chromogenic or fluorogenic substrates can readily be adapted for use in methods provided herein.

Various chromogenic or fluorogenic substrates to produce detectable signals are well known to those skilled in the art and are commercially available. Exemplary substrates that can be utilized to produce a detectable signal include, but are not limited to, 3,3′-diaminobenzidine (DAB), 3,3′,5,5′-tetramethylbenzidine (TMB), chloronaphthol (4-CN)(4-chloro-1-naphthol), 2,2′-azino-bis(3-ethylbenzothiazoline-6-sulphonic acid) (ABTS), o-phenylenediamine dihydrochloride (OPD), and 3-amino-9-ethylcarbazole (AEC) for horseradish peroxidase; 5-bromo-4-chloro-3-indolyl-1-phosphate (BCIP), nitroblue tetrazolium (NBT), Fast Red (Fast Red TR/AS-MX), and p-nitrophenyl phosphate (PNPP) for alkaline phosphatase; 1-methyl-3-indolyl-β-D-galactopyranoside and 2-methoxy-4-(2-nitrovinyl)phenyl β-D-galactopyranoside for β-galactosidase; 2-methoxy-4-(2-nitrovinyl)phenyl β-D-glucopyranoside for β-glucosidase; and the like. Exemplary fluorogenic substrates include, but are not limited to, 4-(trifluoromethyl)umbelliferyl phosphate for alkaline phosphatase; 4-methylumbelliferyl phosphate bis (2-amino-2-methyl-1,3-propanediol), 4-methylumbelliferyl phosphate bis (cyclohexylammonium) and 4-methylumbelliferyl phosphate for phosphatases; QuantaBlu™ and Quintolet for horseradish peroxidase; 4-methylumbelliferyl β-D-galactopyranoside, fluorescein di(β-D-galactopyranoside) and naphthofluorescein di-(β-D-galactopyranoside) for β-galactosidase; 3-acetylumbelliferyl β-D-glucopyranoside and 4-methylumbelliferyl-β-D-glucopyranoside for β-glucosidase; and 4-methylumbelliferyl-α-D-galactopyranoside for α-galactosidase. Exemplary enzymes and substrates for producing a detectable signal are also described, for example, in US publication 2012/0100540. Various detectable enzyme substrates, including chromogenic or fluorogenic substrates, are well known and commercially available (Pierce, Rockford IL; Santa Cruz Biotechnology, Dallas TX; Invitrogen, Carlsbad CA; 42 Life Science; Biocare). Generally, the substrates are converted to products that form precipitates that are deposited at the site of the target nucleic acid. Other exemplary substrates include, but are not limited to, HRP-Green (42 Life Science), Betazoid DAB, Cardassian DAB, Romulin AEC, Bajoran Purple, Vina Green, Deep Space Black™, Warp Red™, Vulcan Fast Red and Ferangi Blue from Biocare (Concord CA; biocare.net/products/detection/chromogens).

Exemplary rare earth metals and metal isotopes suitable as a detectable label include, but are not limited to, lanthanide (III) isotopes such as ¹⁴¹Pr, ¹⁴²Nd, ¹⁴³Nd, ¹⁴⁴Nd, ¹⁴⁵Nd, ¹⁴⁶Nd, ¹⁴⁷Sm, ¹⁴⁸Nd, ¹⁴⁹Sm, ¹⁵⁰Nd, ¹⁵¹Eu, ¹⁵2Sm, ¹⁵³Eu, ¹⁵⁴Sm, ¹⁵⁵Gd, ¹⁵⁶Gd, ¹⁵⁸Gd, ¹⁵⁹Tb ¹⁶⁰Gd, ¹⁶¹Dy, ¹⁶²Dy, ¹⁶³Dy, ¹⁶⁴Dy, ¹⁶⁵Ho, ¹⁶⁶Er, ¹⁶⁷Er, ¹⁶⁸Er, ¹⁶⁹Tm, ¹⁷⁰Er, ¹⁷¹Y, ¹⁷²Yb, ¹⁷³Y, ¹⁷⁴Y, ¹⁷⁵Lu, and ¹⁷⁶Yb. Metal isotopes can be detected, for example, using time-of-flight mass spectrometry (TOF-MS) (for example, Fluidigm Helios and Hyperion systems, fluidigm.com/systems; South San Francisco, CA).

Biotin-avidin (or biotin-streptavidin) is a well-known signal amplification system based on the fact that the two molecules have extraordinarily high affinity to each other and that one avidin/streptavidin molecule can bind four biotin molecules. Antibodies are widely used for signal amplification in immunohistochemistry and ISH. Tyramide signal amplification (TSA) is based on the deposition of a large number of haptenized tyramide molecules by peroxidase activity. Tyramine is a phenolic compound. In the presence of small amounts of hydrogen peroxide, immobilized horseradish peroxidase (HRP) converts the labeled substrate into a short-lived, extremely reactive intermediate. The activated substrate molecules then very rapidly react with and covalently bind to electron-rich moieties of proteins, such as tyrosine, at or near the site of the peroxidase binding site. In this way, many hapten molecules conjugated to tyramide can be introduced at the hybridization site in situ. Subsequently, the deposited tyramide-hapten molecules can be visualized directly or indirectly. Such a detection system is described in more detail, for example, in U.S. publication 2012/0100540.

Embodiments described herein can utilize enzymes to generate a detectable signal using appropriate chromogenic or fluorogenic substrates. It is understood that, alternatively, a label probe can have a detectable label directly coupled to the nucleic acid portion of the label probe. Exemplary detectable labels are well known to those skilled in the art, including but not limited to chromogenic or fluorescent labels (see Hermanson, Bioconjugate Techniques, Academic Press, San Diego (1996)). Exemplary fluorophores useful as labels include, but are not limited to, rhodamine derivatives, for example, tetramethylrhodamine, rhodamine B, rhodamine 6G, sulforhodamine B, Texas Red (sulforhodamine 101), rhodamine 110, and derivatives thereof such as tetramethylrhodamine-5-(or 6), lissamine rhodamine B, and the like; 7-nitrobenz-2-oxa-1,3-diazole (NBD); fluorescein and derivatives thereof; naphthalenes such as dansyl (5-dimethylaminonapthalene-1-sulfonyl); coumarin derivatives such as 7-amino-4-methylcoumarin-3-acetic acid (AMCA), 7-diethylamino-3-[(4′-(iodoacetyl)amino)phenyl]-4-methylcoumarin (DCIA), Alexa fluor dyes (Molecular Probes), and the like; 4,4-difluoro-4-bora-3a,4a-diaza-s-indacene (BODIPY™) and derivatives thereof (Molecular Probes; Eugene, OR); pyrenes and sulfonated pyrenes such as Cascade Blue™ and derivatives thereof, including 8-methoxypyrene-1,3,6-trisulfonic acid, and the like; pyridyloxazole derivatives and dapoxyl derivatives (Molecular Probes); Lucifer Yellow (3,6-disulfonate-4-amino-naphthalimide) and derivatives thereof; CyDye™ fluorescent dyes (Amersham/GE Healthcare Life Sciences; Piscataway NJ), ATTO 390, DyLight 395XL, ATTO 425, ATTO 465, ATTO 488, ATTO 490LS, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thiol2, ATTO Rho101, ATTO 590, ATTO 594, ATTO Rho13, ATTO 610, ATTO 620, ATTO Rho14, ATTO 633, ATTO 643, ATTO 647, ATTO 647N, ATTO 655, ATTO Oxa12, ATTO 665, ATTO 680, ATTO 700, ATTO 725, ATTO 740, Cyan 500 NHS-Ester (ATTO-TECH, Siegen, Germany), and the like. Exemplary chromophores include, but are not limited to, phenolphthalein, malachite green, nitroaromatics such as nitrophenyl, diazo dyes, dabsyl (4-dimethylaminoazobenzene-4′-sulfonyl), and the like.

As disclosed herein, the methods provided herein can be used for concurrent detection of multiple target nucleic acids. In the case of using fluorophores as labels, the fluorophores to be used for detection of multiple target nucleic acids are selected so that each of the fluorophores are distinguishable and can be detected concurrently in the fluorescence microscope in the case of concurrent detection of target nucleic acids. Such fluorophores are selected to have spectral separation of the emissions so that distinct labeling of the target nucleic acids can be detected concurrently. Methods of selecting suitable distinguishable fluorophores for use in methods of the disclosure are well known in the art (see, for example, Johnson and Spence, “Molecular Probes Handbook, a Guide to Fluorescent Probes and Labeling Technologies, 11th ed., Life Technologies (2010)).

Well known methods such as microscopy, cytometry (e.g., mass cytometry, cytometry by time of flight (CyTOF), flow cytometry), or spectroscopy can be utilized to visualize chromogenic, fluorescent, or metal detectable signals associated with the respective target nucleic acids. In general, either chromogenic substrates or fluorogenic substrates, or chromogenic or fluorescent labels, or rare earth metal isotopes, will be utilized for a particular assay, if different labels are used in the same assay, so that a single type of instrument can be used for detection of nucleic acid targets in the same sample.

As disclosed herein, the label can be designed such that the labels are optionally cleavable. As used herein, a cleavable label refers to a label that is attached or conjugated to a label probe so that the label can be removed, for example, in order to use the same label in a subsequent round of labeling and detecting of target nucleic acids. Generally, the labels are conjugated to the label probe by a chemical linker that is cleavable. Methods of conjugating a label to a label probe so that the label is cleavable are well known to those skilled in the art (see, for example, Hermanson, Bioconjugate Techniques, Academic Press, San Diego (1996); Daniel et al., BioTechniques 24(3):484-489 (1998)). One particular system of labeling oligonucleotides is the FastTag™ system (Daniel et al., supra, 1998; Vector Laboratories, Burlinghame CA). Various cleavable moieties can be included in the linker so that the label can be cleaved from the label probe. Such cleavable moieties include groups that can be chemically, photochemically or enzymatically cleaved. Cleavable chemical linkers can include a cleavable chemical moiety, such as disulfides, which can be cleaved by reduction, glycols or diols, which can be cleaved by periodate, diazo bonds, which can be cleaved by dithionite, esters, which can be cleaved by hydroxylamine, sulfones, which can be cleaved by base, and the like (see Hermanson, supra, 1996). One particularly useful cleavable linker is a linker containing a disulfide bond, which can be cleaved by reducing the disulfide bond. In other embodiments, the linker can include a site for cleavage by an enzyme. For example, the linker can contain a proteolytic cleavage site. Generally, such a cleavage site is for a sequence-specific protease. Such proteases include, but are not limited to, human rhinovirus 3C protease (cleavage site LEVLFQ/GP), enterokinase (cleavage site DDDDK/), factor Xₐ (cleavage site IEGR/), tobacco etch virus protease (cleavage site ENLYFQ/G), and thrombin (cleavage site LVPR/GS) (see, for example, Oxford Genetics, Oxford, UK). Another cleavable moiety can be, for example, uracil-DNA (DNA containing uracil), which can be cleaved by uracil-DNA glycosylase (UNG) (see, for example, Sidorenko et al., FEBS Lett. 582(3):410-404 (2008)).

The cleavable labels can be removed by applying an agent, such as a chemical agent or light, to cleave the label and release it from the label probe. As discussed above, useful cleaving agents for chemical cleavage include, but are not limited to, reducing agents, periodate, dithionite, hydroxylamine, base, and the like (see Hermanson, supra, 1996). One useful method for cleaving a linker containing a disulfide bond is the use of tris(2-carboxyethyl)phosphine (TCEP) (see Moffitt et al., Proc. Natl. Acad. Sci. USA 113:11046-11051 (2016)). In one embodiment, TCEP is used as an agent to cleave a label from a label probe.

In some embodiments, the method for detecting a target nucleic acid in a cell provided herein comprises a pretreatment step before hybridization of the target probe(s). In some embodiments, the pretreatment step comprises a blocking step where certain blocking agent(s) is/are applied to block certain endogenous components of the cell thus reducing assay background. For example, hydrogen peroxide is a blocking agent when horseradish peroxidase (HRP) is used as detection enzyme in the later steps. Hydrogen peroxide is added to inactivate the endogenous HRP activity in the sample, thus reducing assay background. In a specific embodiment, this blocking step is added as the first step in the pretreatment right after deparaffinization. In some embodiments, the pretreatment step comprises an epitope retrieval step, where certain epitope retrieval buffer(s) can be added to unmask the target nucleic acid. In some embodiments, the epitope retrieval step comprises heating the sample. In some embodiments, the epitope retrieval step comprises heating the sample to 50° C. to 100° C. In one embodiment, the epitope retrieval step comprises heating the sample to about 88° C. In some embodiments, the pretreatment step comprises a permeabilization step to retain the nucleic acid targets in the cell and to permit the target probe(s), signal-generating complex, etc. to enter the cell. In some embodiments, the permeabilization step comprises a digestion with a protease. Detergents (e.g., Triton X-100 or SDS) and Proteinase K can also be used to increase the permeability of the fixed cells. Detergent treatment, usually with Triton X-100 or SDS, is frequently used to permeate the membranes by extracting the lipids. Proteinase K is a nonspecific protease that is active over a wide pH range and is not easily inactivated. It is used to digest proteins that surround the target mRNA. Optimal concentrations and durations of treatment can be experimentally determined as is well known in the art. A cell washing step can follow, to remove the dissolved materials produced in the any steps in the pretreatment step. In some embodiments, the sample is in a formalin-fixed paraffin embedded tissue, a deparaffinization step is needed, when paraffin is removed.

For methods of the present disclosure, cells are optionally fixed and/or permeabilized before hybridization of the target probes. Fixing and permeabilizing cells can facilitate retaining the nucleic acid targets in the cell and permit the target probes, label probes, and so forth, to enter the cell and reach the target nucleic acid molecule. The cell is optionally washed to remove materials not captured to a nucleic acid target. The cell can be washed after any of various steps, for example, after hybridization of the target probes to the nucleic acid targets to remove unbound target probes, and the like. Methods for fixing and permeabilizing cells for in situ detection of nucleic acids, as well as methods for hybridizing, washing and detecting target nucleic acids, are also well known in the art (see, for example, US 2008/0038725; US 2009/0081688; Hicks et al., J. Mol. Histol. 35:595-601 (2004); Stoler, Clinics in Laboratory Medicine 10(1):215-236 (1990); In situ hybridization. A practical approach, Wilkinson, ed., IRL Press, Oxford (1992); Schwarzacher and Heslop-Harrison, Practical in situ hybridization, BIOS Scientific Publishers Ltd, Oxford (2000); Shapiro, Practical Flow Cytometry 3rd ed., Wiley-Liss, New York (1995); Ormerod, Flow Cytometry, 2nd ed., Springer (1999)). Exemplary fixing agents include, but are not limited to, aldehydes (formaldehyde, glutaraldehyde, and the like), acetone, alcohols (methanol, ethanol, and the like), formal calcium, formal saline, zinc formalin, Zenker's fixative, Helly's fixative, B-5 fixative, Bouin's solution, Hollande's fixative, Gendre's solution, Clarke's solution, Carnoy's solution, Methacarn, Alcoholic formalin, formol acetic alcohol, and I.B.F. tissue fixative. Exemplary permeabilizing agents include, but are not limited to, alcohols (methanol, ethanol, and the like), acids (glacial acetic acid, and the like), detergents (Triton, NP-40, Tween™ 20, and the like), saponin, digitonin, Leucoperm™ (BioRad, Hercules, CA), and enzymes (for example, lysozyme, lipases, proteases and peptidases). Permeabilization can also occur by mechanical disruption, such as in tissue slices.

For in situ detection of double stranded nucleic acids, generally the sample is treated to denature the double stranded nucleic acids in the sample to provide accessibility for the target probes to bind by hybridization to both strands of the target double stranded nucleic acid. Conditions for denaturing double stranded nucleic acids are well known in the art, and include heat and chemical denaturation, for example, with base (NaOH), formamide, dimethyl sulfoxide, and the like (see Wang et al., Environ. Health Toxicol. 29:e2014007 (doi: 10.5620/eht.2014.29.e2014007) 2014; Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, MD (1999)). For example, NaOH, LiOH or KOH, or other high pH buffers (pH>11) can be used to denature double stranded nucleic acids such as DNA. In addition, heat and chemical denaturation methods can be used in combination.

The methods of detecting DNA described herein can be used, for example, in physical mapping of DNA sequences in chromosomes; three dimensional (3D) mapping of spatial genome organization; detection of gene copy number gain (duplication and amplification), loss (deletion) and gene rearrangement (translocation and fusion) in diseased cells and tissues; prenatal, postnatal and pre-transplantation diagnosis of chromosomal abnormalities; cancer diagnosis and prognosis; companion diagnostics; and detection and identification of pathogens (for example, bacteria and viruses).

### 4. Compositions

Embodiments of the present disclosure also include a composition for use in a method of removing RNA in a sample, the composition comprising sodium metasilicate. In some embodiments, the disclosure provides a composition comprising: (i) sodium metasilicate; and (ii) a sample comprising plurality of cells.

In some embodiments, the sample comprises cultured cells. In some embodiments, the sample is a tissue specimen or is derived from a tissue specimen. In some embodiments, the sample is a blood sample or is derived from a blood sample. In some embodiments, the sample is a cytological sample or is derived from a cytological sample. In another embodiment, the biological sample is an exosome. Various samples and methods of obtaining and/or processing samples are described herein.

In some embodiments, the composition further includes one or more components useful for carrying out a nucleic acid hybridization reaction, such as an in situ hybridization reaction or a hybridization chain reaction assay. For example, the composition can include one or more of a hybridization buffer, dextran sulfate, formamide, dithiothreitol (DDT), sodium chloride and sodium citrate (SSC), EDTA, Denhardt's solution, a fluorescent label, a chromogenic label, dNTPs, single-stranded DNA, tRNA, polyA, an initiator oligo, or any combination thereof.

In some embodiments, the composition further includes one or more probes for detecting DNA in the sample. The probe can be a probe described herein, e.g., a set of one or more target probes described herein. In some embodiments, the composition further includes an SGC, such as an SGC described herein, e.g., one which includes a label probe, and optionally, one or more of an amplifier, a pre-amplifier, and a pre-pre-amplifier. In some embodiments, the label probe includes at least one detectable label.

### 5. Kits

Embodiments of the present disclosure also include a kit for removing RNA from a sample, wherein the kit comprises sodium metasilicate. Embodiments of the present disclosure also include a kit for detecting a target DNA in a sample, comprising sodium metasilicate and one or more probes or reagents for detecting the target DNA in the sample.

In some embodiments, the kit comprises an aqueous solution of sodium metasilicate. The aqueous solution may have a concentration of sodium metasilicate of about 50 mM to about 200 mM, for example, about 50 mM, about 55 mM, about 60 mM, about 65 mM, about 70 mM, about 75 mM, about 80 mM, about 85 mM, about 90 mM, about, 95 mM, about 100 mM, about, 105 mM, about 110 mM, about 115 mM, about 120 mM, about 125 mM, about 130 mM, about 135 mM, about 140 mM, about 145 mM, about 150 mM, about 155 mM, about 160 mM, about 165 mM, about 170 mM, about 175 mM, about 180 mM, about 185 mM, about 190 mM, about 195 mM, or about 200 mM. In some embodiments, the aqueous solution has a concentration of sodium metasilicate of about 100 mM.

In some embodiments, the aqueous solution of sodium metasilicate has a pH of about 12 to about 14, or about 12.5 to about 13.5, or about 12.5 to about 13.0. For example, in some embodiments, the aqueous solution of sodium metasilicate has a pH of about 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, or 14.0. In some embodiments, the aqueous solution of sodium metasilicate has a pH of about 12.8.

In some embodiments, the kit further comprises a probe for detecting the target DNA in the sample, e.g., a set of one or more target probes described herein. In some embodiments, the kit also includes an SGC, such as an SGC described herein. In some embodiments, the SGC includes a label probe, and optionally, one or more of an amplifier, a pre-amplifier, and a pre-pre-amplifier. In some embodiments, the label probe includes at least one detectable label. In one embodiment, the kit provided herein comprises agents for performing RNAscope® as described in more detail in, e.g., U.S. Pat. Nos. 7,709,198, 8,604,182, and 8,951,726, which are herein incorporated by reference in their entireties. In another embodiment, the kit provided herein comprises agents for performing BaseScope™, which is described in more detail in, e.g., US Patent Publication No. 2013/0171621, and PCT Publication No. WO 2011/094669. In some embodiments, the kit comprises at least one set of two or more target probes capable of hybridizing to a target nucleic acid, and an SGC capable of hybridizing to the set of two or more target probes.

In some embodiments, the kit further includes other agents or materials for performing a DNA in situ hybridization assay, including but not limited to, fixing agents and agents for treating samples for preparing hybridization, agents for washing samples, and the like. In some embodiments, the kit includes at least one of a hybridization buffer, dextran sulfate, formamide, dithiothreitol (DDT), sodium chloride and sodium citrate (SSC), EDTA, Denhardt's solution, a fluorescent label, a chromogenic label, dNTPs, single-stranded DNA, tRNA, polyA, an initiator oligo, or any combination thereof.

In some embodiments, the kit further includes a calibrator or control polynucleotide.

The kits of the present disclosure may further include instructions and/or packaging material, which generally includes a physical container for housing and/or delivering the components of the kit. The packaging material can maintain the components sterilely, and can be made of material commonly used for such purposes (e.g., paper, corrugated fiber, glass, plastic, foil, ampules, vials, tubes, etc.). In some embodiments, the kit comprises instructions for removing RNA from a sample. In some embodiments, the kit comprises instructions for carrying out a DNA in situ hybridization assay.

Kits provided herein can include labels or inserts, such as instructions for performing an assay.

Labels or inserts can include “printed matter,” e.g., paper or cardboard, separate or affixed to a component, a kit or packing material (e.g., a box), or attached to, for example, an ampule, tube or vial containing a kit component. Labels or inserts can additionally include a computer readable medium, such as a disk (e.g., hard disk, card, and memory disk), optical disk such as CD- or DVD-ROM/RAM, DVD, MP3, magnetic tape, or an electrical storage media such as RAM and ROM or hybrids of these such as magnetic/optical storage media, FLASH media, or memory type cards. Labels or inserts can include information identifying manufacturer information, lot numbers, manufacturer location, and date.

### 6. Examples

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure.

### Example 1

### RNA Removal Using RNase A

RNA was removed from samples of HEK293 cells using the traditional enzymatic approach with RNase A. The RNase A solution (Ribonuclease A from bovine pancrease (Sigma cat #4246) was diluted (1:100) in 1×PBS and then applied to the samples after the protease pretreatment step and prior to probe hybridization. The samples were then incubated at 40° C. for 30 min. The RNase A solution was then removed from the slides with two water washes followed by probe addition and incubation and using RNAscope ISH assay for signal detection. The signal was detected using fast red chromogen (BioCare) followed by hematoxylin counterstaining.

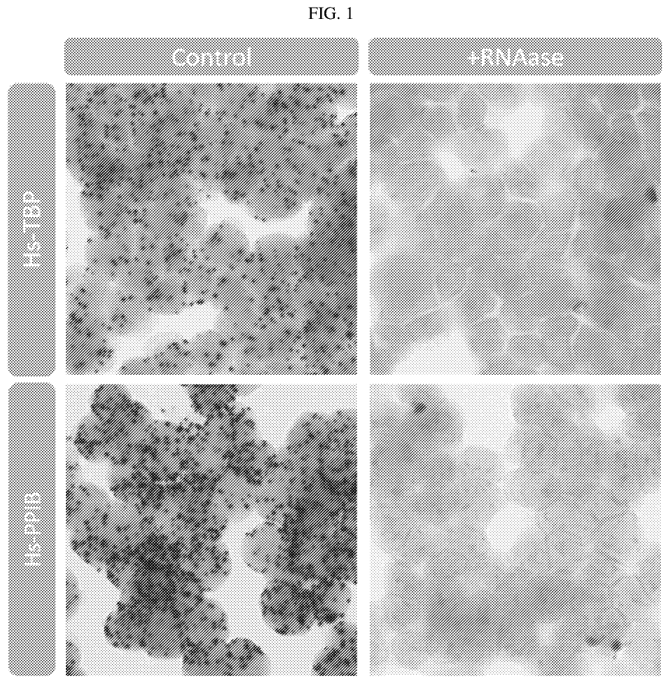

Images of cells are shown in FIG. 1, demonstrating the staining pattern using the RNAscope™ assay for two RNA targets: TBP (TATA-Box Binding Protein, a gene with relatively low constitutive expression) and PPIB (peptidylprolyl isomerase B, a gene with relatively high constitutive expression) on HEK293 cytospin samples. The figure illustrates the successful removal of both TBP and PPIB RNA signal with RNase A application, but also shows the degraded morphology of the cells in both examples

### Example 2

### RNA Removal Using Sodium Metasilicate

A 100 mM sodium metasilicate solution (pH 12.8) was added to FFPE samples after deparaffinization, and incubated at 40° C. for 45 minutes. This step was followed by two rounds of water washes. The slides were then transferred onto a Leica bond RX instrument and RNAscope® 2.5 LS Assay-RED (https://acdbio.com/rnascope%C2%AE-25-ls-assay-red) was used for signal detection followed by hematoxylin counter staining.

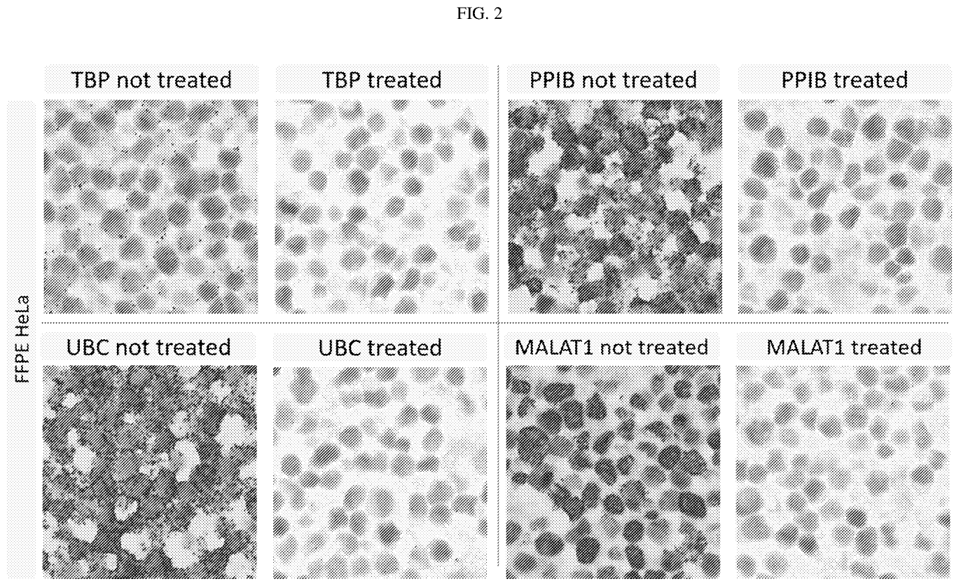

The samples were stained with various RNA markers having low to very high expression levels: TBP (TATA Binding Protein, low expresser), PPIB (Peptidylprolyl Isomerase B, high expresser), UBC (Ubiquitin C, high expresser) and MALAT1 (Metastasis Associated Lung Adenocarcinoma Transcript 1, very high expresser). Images are shown in FIG. 2. Even after treatment with sodium metasilicate and removal of almost all RNA molecules, the nuclear and cellular morphology of the samples are unaffected, and the morphological integrity of all samples are intact and comparable to that of the untreated samples.

### Example 3

### Detection of DNA Using an RNAscope-Based DNA ISH Assay

A 100 mM sodium metasilicate solution (pH 12.8) was added to FFPE samples after deparaffinization, and incubated at 40° C. for 45 minutes. This step was followed by two rounds of water washes. The slides were then transferred onto a Leica bond RX instrument and a modified RNAscope® 2.5 LS Assay-RED (https://acdbio.com/rnascope%C2%AE-25-ls-assay-red) was used for signal detection followed by hematoxylin counter staining

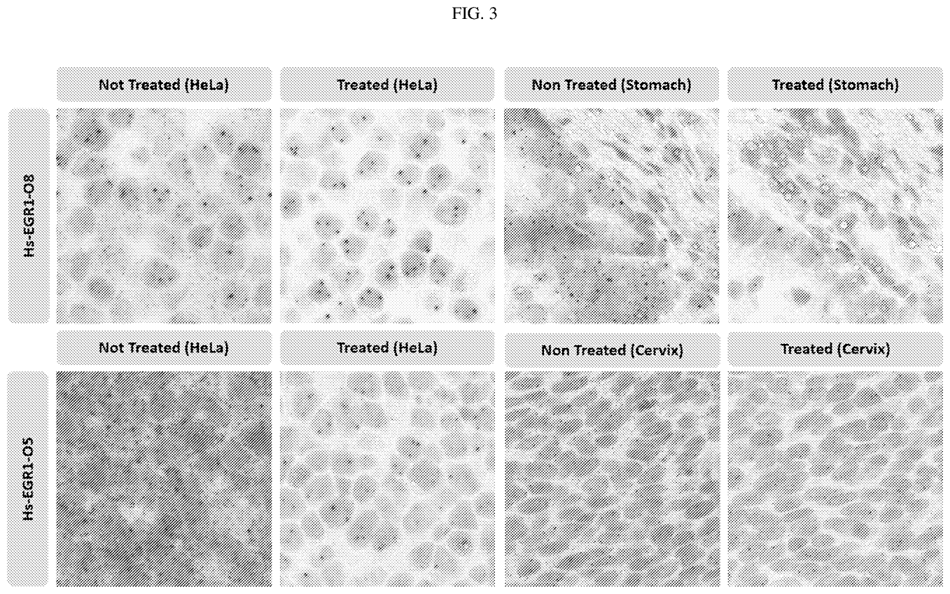

Staining patterns using two sense probes EGR1-O8 and EGR1-O5 for the EGR1 (Early Growth Response 1) gene are shown in FIG. 3. Staining prior to RNA removal procedure and post sodium metasilicate application on both cell line and human tissue samples are shown. Sodium metasilicate successfully removed the RNA molecules from both HeLa samples as well as human tissue that were being cross detected without affecting the DNA molecules.

### Example 4

### Detection of DNA Using a Fluorescent ISH Assay

A 100 mM sodium metasilicate solution (pH 12.8) was added FFPE samples after deparaffinization, and incubated at 40° C. for 45 minutes. This step was followed by two rounds of water washes. The slides were then transferred onto a Leica bond RX instrument and a RNAscope® LS Multiplex Fluorescent Assay (https://acdbio.com/rnascope%C2%AE-ls-multiplex-fluorescent-assay) was used for signal detection followed by DAPI counter staining.

The staining pattern using the RNAscope™ assay before and after RNA removal treatment for the FFPE sample stained with MALAT1 antisense probe is shown. Sodium metasilicate successfully removed RNA molecules from the sample stained with the MALAT1 probe.

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

It is understood that the foregoing detailed description and accompanying examples are merely illustrative and are not to be taken as limitations upon the scope of the disclosure, which is defined solely by the appended claims and their equivalents.

All publications and patents mentioned in the above specification are herein incorporated by reference as if expressly set forth herein. Various changes and modifications to the disclosed embodiments will be apparent to those skilled in the art and may be made without departing from the spirit and scope thereof.

1. A method of removing RNA from a sample, the method comprising:
contacting the sample with an effective amount of sodium metasilicate.

2. The method of claim 1, wherein the sample is a biological sample.

3. The method of claim 2, wherein the sample comprises cultured cells.

4. The method of claim 2, wherein the sample is a tissue specimen or is derived from a tissue specimen.

5. The method of claim 4, wherein the sample is a formalin fixed paraffin embedded tissue specimen.

6. The method of claim 5, wherein the method further comprises performing a deparaffinization step prior to contacting the sample with the sodium metasilicate.

7. The method of claim 2, wherein the sample is a blood sample or is derived from a blood sample.

8. The method of claim 2, wherein the sample is a cytological sample or is derived from a cytological sample.

9. The method of any one of claims 1-8, wherein the sodium metasilicate is in an aqueous solution.

10. The method of claim 9, wherein the concentration of the sodium metasilicate in the solution is about 50 mM to about 200 mM.

11. The method of claim 10, wherein the concentration of the sodium metasilicate in the solution is about 100 mM.

12. The method of any one of claims 9-11, wherein the solution of sodium metasilicate has a pH of about 12 to about 14.

13. The method of claim 12, wherein the solution of sodium metasilicate has a pH of about 12.5 to about 13.0.

14. The method of any one of claims 1-13, further comprising heating the sample after the contacting step.

15. The method of claim 14, wherein the sample is heated to a temperature of about 35° C. to about 45° C.

16. The method of claim 15, wherein the sample is heated to a temperature of about 40° C.

17. The method of any one of claims 14-16, comprising heating the sample for about 30 minutes to about 60 minutes.

18. The method of claim 17, comprising heating the sample for about 45 minutes.

19. The method of any one of claims 1-18, further comprising washing the sample after the contacting step.

20. The method of any one of claims 1-19, further comprising detecting a target DNA in the sample after the contacting step.

21. The method of claim 20, wherein the target DNA is detected by DNA in situ hybridization.

22. The method of any one of claims 1-21, wherein the sample morphology is substantially unchanged following the contacting step.

23. The method of any one of claims 1-22, wherein RNA levels are reduced by at least 90% in the sample.

24. The method of any one of claims 1-23, wherein the RNA removed from the sample comprises mRNA.

25. A method of preparing a biological sample for detection of a target DNA in the sample, comprising:
contacting the sample with sodium metasilicate.

26. The method of claim 25, wherein the biological sample comprises cultured cells.

27. The method of claim 25, wherein the biological sample is a tissue specimen or is derived from a tissue specimen.

28. The method of claim 27, wherein the biological sample is a formalin fixed paraffin embedded tissue specimen.

29. The method of claim 28, wherein the method further comprises performing a deparaffinization step prior to contacting the biological sample with the sodium metasilicate.

30. The method of claim 25, wherein the biological sample is a blood sample or is derived from a blood sample.

31. The method of claim 25, wherein the biological sample is a cytological sample or is derived from a cytological sample.

32. The method of any one of claims 25-31, wherein the sodium metasilicate is in an aqueous solution.

33. The method of claim 32, wherein the concentration of the sodium metasilicate in the solution is about 50 mM to about 200 mM.

34. The method of claim 33, wherein the concentration of the sodium metasilicate in the solution is about 100 mM.

35. The method of any one of claims 31-34, wherein the solution of sodium metasilicate has a pH of about 12 to about 14.

36. The method of claim 35, wherein the solution of sodium metasilicate has a pH of about 12.5 to about 13.0.

37. The method of any one of claims 25-36, further comprising heating the sample after the contacting step.

38. The method of claim 37, wherein the sample is heated to a temperature of about 35° C. to about 45° C.

39. The method of claim 38, wherein the sample is heated to a temperature of about 40° C.

40. The method of any one of claims 36-39, comprising heating the sample for about 30 minutes to about 60 minutes.

41. The method of claim 40, comprising heating the sample for about 45 minutes.

42. The method of any one of claims 25-41, further comprising washing the sample after the contacting step.

43. The method of any one of claims 25-42, further comprising detecting a target DNA in the sample after the contacting step.

44. The method of claim 43, wherein the target DNA is detected by DNA in situ hybridization.

45. The method of any one of claims 25-44, wherein the sample morphology is substantially unchanged following contacting of the sample with the sodium metasilicate.

46. The method of any one of claims 25-45, wherein RNA levels are reduced by at least 90% in the sample.

47. The method of any one of claims 25-46, wherein the RNA removed from the sample comprises mRNA.

48. A composition comprising:
sodium metasilicate; and
a sample comprising a plurality of cells.

49. The composition of claim 48, wherein the sample is a tissue specimen or is derived from a tissue specimen.

50. The composition of claim 49, wherein the sample is a formalin fixed paraffin embedded tissue specimen or is derived from a formalin fixed paraffin embedded tissue specimen.

51. The composition of claim 48, wherein the sample is a blood sample or is derived from a blood sample.

52. The composition of claim 48, wherein the sample is a cytological sample or is derived from a cytological sample.

53. A kit comprising:
sodium metasilicate; and
one or more probes or reagents for detecting a target DNA in a sample.

54. The kit of claim 53, comprising one or more target probes capable of hybridizing to the target DNA in the sample.

55. The kit of claim 53 or claim 54, comprising one or more reagents for detecting DNA in the sample, wherein the reagents are selected from a hybridization buffer, dextran sulfate, formamide, dithiothreitol (DTT), sodium chloride and sodium citrate (SSC), EDTA, Denhardt's solution, a fluorescent label, a chromogenic label, dNTPs, single-stranded DNA, tRNA, polyA, an initiator oligonucleotide, or any combination thereof.

56. The kit of claim 54 or 55, further comprising a signal generating complex capable of hybridizing to the one or more target probes.

57. The kit of claim 56, wherein the signal generating complex comprises a label probe, and optionally, one or more of an amplifier, a pre-amplifier, and a pre-pre-amplifier.

58. The kit of any one of claims 53-57, further comprising a calibrator or control polynucleotide.

59. The kit of any one of claims 53-58, wherein the sodium metasilicate is in an aqueous solution.

60. The kit of claim 59, wherein the concentration of the sodium metasilicate in the solution is about 50 mM to about 200 mM.

61. The kit of claim 60, wherein the concentration of the sodium metasilicate in the solution is about 100 mM.

62. The kit of any one of claims 59-61, wherein the solution of sodium metasilicate has a pH of about 11 to about 14.

63. The kit of claim 62, wherein the solution of sodium metasilicate has a pH of about 12.5 to about 13.0.

64. The kit of any one of claims 53-63, further comprising instructions for carrying out a DNA in situ hybridization assay.

65. A kit comprising:
sodium metasilicate; and
instructions for removing RNA from a sample using the sodium metasilicate.

66. The kit of claim 65, wherein the sodium metasilicate is in an aqueous solution.

67. The kit of claim 65 or claim 66, wherein the concentration of the sodium metasilicate in the solution is about 50 mM to about 200 mM.

68. The kit of claim 67, wherein the concentration of the sodium metasilicate in the solution is about 100 mM.

69. The kit of any one of claims 65-68, wherein the solution of sodium metasilicate has a pH of about 11 to about 14.

70. The kit of claim 69, wherein the solution of sodium metasilicate has a pH of about 12.5 to about 13.0.
